(19) United States
(12) Patent Application Publication
(10) Pub. No.: US 2009/0253746 A1
(43) Pub. Date: Oct. 8, 2009

(54) Synthesis and Preparations of Intermediates and New Polymorphs Thereof Useful For The Preparation Of Donepezil Hydrochlcoride

(76) Inventor: Nuria Soldevilla Madrid, Barcelona (ES)

Correspondence Address: HOGAN & HARTSON LLP;IP GROUP, COLUMBIA SQUARE, 555 THIRTEENTH STREET, N.W., WASHINGTON, DC, 20004, US

(21) Appl. No.: 12/084,942

(22) PCT Filed: Nov. 14, 2006
(86) PCT No.: PCT/IB2006/004254

§ 371 Date: May 18, 2009

Related U.S. Application Data
(60) Provisional application No. 60/735,838, filed on Nov. 14, 2005.

(51) Int. Cl. A61K 31/445; C07D 211/06

(52) U.S. Cl. 514/319; 546/206

(57) ABSTRACT

The invention relates to an improved process for preparing 2-(1-benzylpiperidin-4-ylmethyliden)-5,6-dimethoxyin-dan-1-one (a key intermediate in the synthesis of donepezil hydrochloride), crystalline polymorph forms of this key intermediate and their use thereof for producing donepezil hydrochloride. In particular, the invention provides an improved method for producing the intermediate 2-(1-benzylpiperidin-4-ylmethyliden)-5,6-dimethoxyindan-1-one. The process includes reacting 5,6-dimethoxyin-dan-1-one with 1-benzylpiperidine-4-carbaldehyde using potassium hydroxide in an aqueous solvent. The aqueous solvent can be a mixture of an organic solvent and water Where the organic solvent is not miscible with water, the reaction may be performed in the presence of a phase transfer catalyst.

### CROSS REFERENCE TO RELATED APPLICATIONS

This application claims priority to U.S. Provisional Application No. 60/735,838, filed Nov. 14,2005, which is expressly incorporated herein by reference in its entirety.

### BACKGROUND OF THE INVENTION

1. Field of the Invention

The invention relates to an improved process for preparing 2-(1-benzylpiperidin-4-ylmethyliden)-5,6-dimethoxyindan-1-one (a key intermediate in the synthesis of donepezil hydrochloride), crystalline polymorph forms of this key intermediate and their use thereof for producing donepezil hydrochloride.

2. Discussion of the Related Art

Donepezil hydrochloride is a commercially marketed, pharmaceutically active ingredient prescribed for the treatment of mild to moderate dementia of the Alzheimer's type. Donepezil hydrochloride is also known as 2,3-dihydro-5,6-dimethoxy-2-[[1-(phenylmethyl)-4-piperidinyl]methyl-1H-inden-1-one hydrochloride or 2-(1-benzyl-piperidin-4-ylmethyl)-5,6-dimethoxy-indan-1-one and has the following structure:

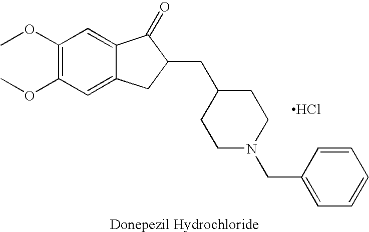

Donepezil hydrochloride is an orally active reversible inhibitor of the enzyme acetylcholinesterase and is marketed under the name ARICEPT®.

According to each of U.S. Pat. No. 4,895,841, WO 9839000 A1, EP 0296560 B1 and J. Med. Chem., 1995, 38, 4821-9, donepezil hydrochloride can be prepared by hydrogenation of the intermediate 2-(1-benzylpiperidin-4-ylmethyliden)-5,6-dimethoxyindan-1-one (Scheme 1, Compound 1) with Pd/C followed by treatment with hydrochloric acid. Compound 1 can be obtained by reacting 5,6-dimethoxyindan-1-one (Scheme 1, Compound 2) and 1-benzylpiperidine-4-carbaldehyde (Scheme 1, Compound 3) and using LDA (generated in situ by reaction of N,N-diisopropylamine and n-butyllithium) in a mixture of tetrahydrofuran and hexamethylphosphoramide (HMPA) at −78° C.

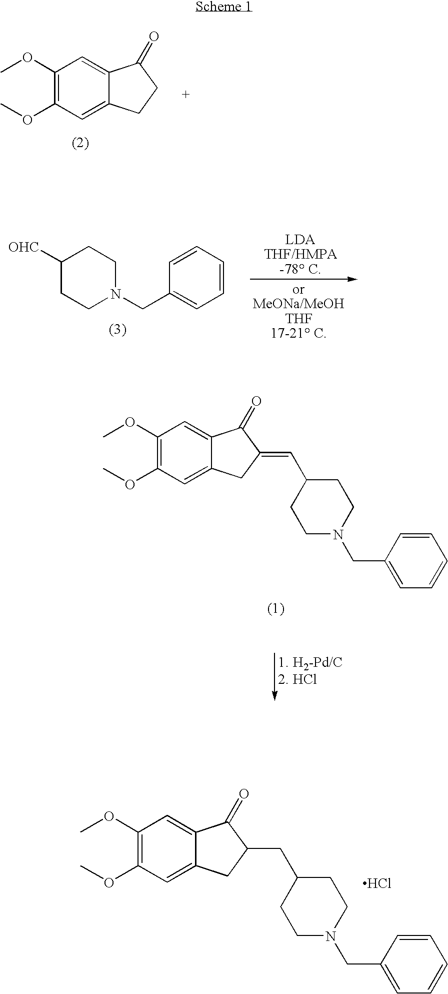

### SUMMARY OF THE INVENTION

The invention relates to an improved process for preparing 2-(1-benzylpiperidin-4-ylmethyliden)-5,6-dimethoxyindan-1-one (a key intermediate in the synthesis of donepezil hydrochloride), crystalline polymorph forms of this key intermediate and their use thereof for producing donepezil hydrochloride.

In particular, the invention provides an improved method for producing the intermediate 2-(1-benzylpiperidin-4-ylmethyliden)-5,6-dimethoxyindan-1-one (Scheme 1, Compound 1). The process includes reacting 5,6-dimethoxyindan-1-one (Scheme 1, Compound 2) with 1-benzylpiperidine-4-carbaldehyde (Scheme 1, Compound 3) using potassium hydroxide in an aqueous solvent. The aqueous solvent can be a mixture of an organic solvent and water. Where the organic solvent is not miscible with water, the reaction may be performed in the presence of a phase transfer catalyst.

As such, this method avoids the use of dangerous and/or toxic chemicals, such as n-butyllithium and HMPA, and is therefore less hazardous than previously described processes. Additionally, the improved method is performed without the need to use a powerful cooling system to decrease the temperature of the reaction to −78° C. as required in the previously described processes.

The invention further includes providing new polymorphic forms of the intermediate 2-(1-benzylpiperidin-4-ylmethyliden)-5,6-dimethoxyindan-1-one (Scheme 1, Compound 1) as well as providing the same in high purity.

The invention further includes providing the intermediate 2-(1-benzylpiperidin-4-ylmethyliden)-5,6-dimethoxyindan-1-one (Scheme 1, Compound 1) with low or no quantities of synthetic by-products (e.g., its regioisomers and 2-[(1-benzylpiperidin-4-yl)hydroxymethyl]-5,6-dimethoxyindan-1-one (Compound 4, Table 1).

The invention further includes preparing donepezil hydrochloride from 2-(1-benzylpiperidin-4-ylmethyliden)-5,6-dimethoxyindan-1-one prepared according to this new process as well as from these new polymorphic forms of 2-(1-benzylpiperidin-4-ylmethyliden)-5,6-dimethoxyindan-1-one.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention. In the drawings:

FIG. 1 illustrates an X-ray powder diffractogram of Form I of 2-(1-benzylpiperidin-4-ylmethyliden)-5,6-dimethoxyindan-1-one (Scheme 1, Compound 1);

FIG. 2 illustrates an X-ray powder diffractogram of Form II of 2-(1-benzylpiperidin-4-ylmethyliden)-5,6-dimethoxyindan-1-one (Scheme 1, Compound 1); and

FIG. 3 illustrates an X-ray powder diffractogram of Form I of donepezil hydrochloride.

### DETAILED DESCRIPTION OF THE PREFERRED EMBODIMENTS

Reference will now be made in detail to the preferred embodiments of the invention. This invention may, however, be embodied in many different forms and should not be construed as limited to the embodiments set forth herein. In addition, and as will be appreciated by one of skill in the art, the invention may be embodied as a method, system or process.

The invention relates to an improved process for preparing 2-(1-benzylpiperidin-4-ylmethyliden)-5,6-dimethoxyindan-1-one (a key intermediate in the synthesis of donepezil hydrochloride), crystalline polymorph forms of this key intermediate and the use thereof for producing donepezil hydrochloride.

In particular, one aspect of the invention includes a process for preparing 2-(1-benzylpiperidin-4-ylmethyliden)-5,6-dimethoxyindan-1-one (Scheme 1, Compound 1), a key intermediate in the synthesis of donepezil hydrochloride. The process of preparing Compound 1 includes reacting 5,6-dimethoxyindan-1-one (Scheme 1, Compound 2) with 1-benzylpiperidine-4-carbaldehyde (Scheme 1, Compound 3) using an alkali metal hydroxide in a mixture of an organic solvent and water at a temperature between room temperature and 120° C. The reaction may optionally be carried out in the presence of a phase transfer catalyst.

Another aspect of the invention includes a process for preparing Compound 1 that includes reacting Compound 2 with Compound 3 using potassium hydroxide in a mixture of toluene and water at reflux temperature (˜93-95° C.) in the presence of a phase transfer catalyst (e.g., benzyltriethylammonium chloride).

Another aspect of the invention includes a process for preparing Compound 1 that includes reacting Compound 2 with Compound 3 using potassium hydroxide in a mixture of tetrahydrofuran and water.

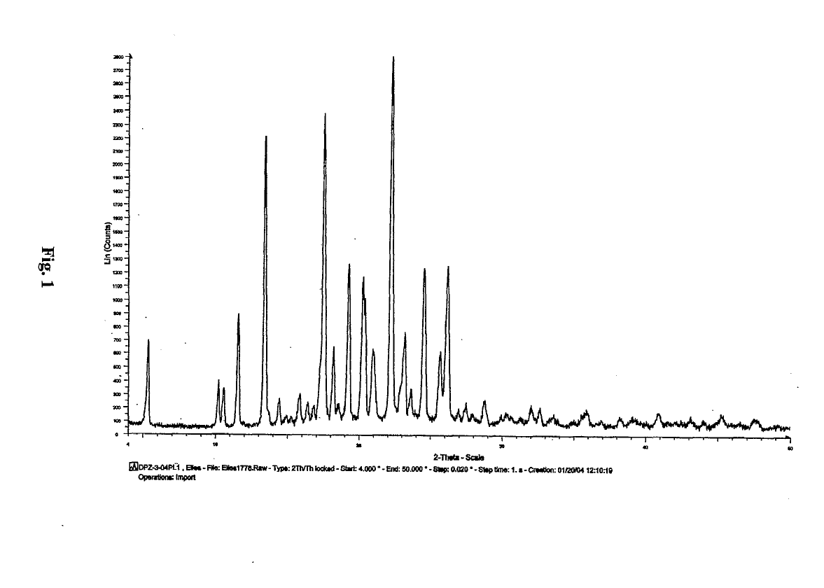

Another aspect of the invention includes a solid, crystalline polymorph of Compound 1, designated as Form I, having an X-ray diffraction pattern substantially similar to that of FIG. 1. This polymorph is obtained when the reaction of Compounds 2 and 3 is performed in a mixture of tetrahydrofuran and water, and the resulting product is isolated by filtration from water after removing the tetrahydrofuran by distillation.

Another aspect of the invention includes isolating solid, crystalline polymorph Form I of Compound 1 by filtration from water after removing the tetrahydrofuran by distillation.

Another aspect of the invention includes solid, crystalline polymorph Form I of Compound 1 having an X-ray diffraction pattern (2θ) having characteristic peaks at 5.28, 10.52, 11.54, 13.40, 17.51, 18.17, 19.24, 20.24, 20.95, 22.23, 23.15, 24.52, 25.64, 26.16 degrees.

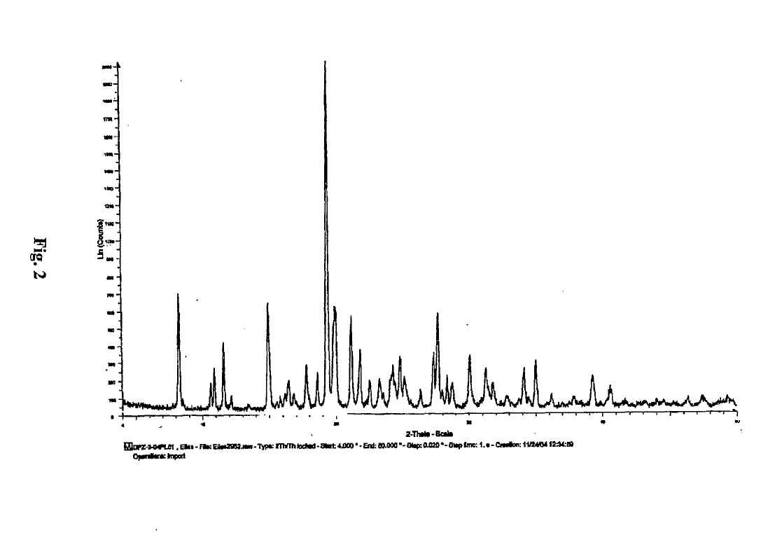

Another aspect of the invention includes a solid, crystalline polymorph of Compound 1, designated as Form II, having an X-ray diffraction pattern substantially similar to that of FIG. 2. This polymorph is obtained when the reaction of Compounds 2 and 3 is performed in a mixture of toluene and water, and the resulting product is isolated by filtration after cooling the reaction mixture.

Another aspect of the invention includes isolating solid, crystalline polymorph Form II of Compound 1 by filtration from toluene/water after cooling the reaction mixture.

Another aspect of the invention includes purifying crystalline Compound 1 by treatment with water and/or isopropyl alcohol.

Another aspect of the invention includes solid, crystalline polymorph Form II of Compound 1 having an X-ray diffraction pattern (2θ) having characteristic peaks at 8.17, 11.51, 14.87, 17.68, 19.29, 19.91, 21.09, 21.74, 24.75, 27.62 degrees.

Another aspect of the invention includes using solid, crystalline polymorph Form I of Compound 1 for preparing donepezil hydrochloride.

Another aspect of the invention includes using solid, crystalline polymorph Form II of Compound 1 for preparing donepezil hydrochloride.

Another aspect of the invention includes solid, crystalline Compound 1, Form I, Form II and mixtures thereof, having a purity higher than 95.0%, higher than 98.0% and/or higher than 98.9% as measured by high performance liquid chromatography and the use of the same for preparing donepezil hydrochloride.

Another aspect of the invention includes solid, crystalline Compound 1, Form I, Form II or mixtures thereof, having a content of 2-[(1-benzylpiperidin-4-yl)hydroxymethyl]-5,6-dimethoxyindan-1-one (Compound 4) of less than 2.5%, of less than 1.0% and/or less than 0.05% as measured by high performance liquid chromatography and the use of the same for preparing donepezil hydrochloride.

Another aspect of the invention includes solid, crystalline Compound 1, Form I, Form II or mixtures thereof, having a content of its corresponding regioisomer of less than 3.0% and/or less than 1.5% and the use of the same for preparing donepezil hydrochloride.

It will be apparent to those skilled in the art that various modifications and variations can be made in the invention and specific examples provided herein without departing from the spirit or scope of the invention. Thus, it is intended that the invention covers the modifications and variations of this invention that come within the scope of any claims and their equivalents.

The following examples are for illustrative purposes only and are not intended, nor should they be interpreted to, limit the scope of the invention.

### EXAMPLES

### General Experimental Conditions:

### HPLC Method

Chromatographic separation was carried out using a Waters XTerra MS C18, 5 μm, 15 cm×4.6 mm. I.D column.

The mobile phase A was 0.01 M ammonium bicarbonate (NH₄HCO₃) buffer (pH=7.0) which was prepared from 0.79 g of NH₄HCO₃ dissolved in 1000 mL of water. The pH was adjusted to 7.0 with formic acid. The mobile phase was mixed and filtered through a 0.22 μm nylon filter under vacuum.

The mobile phase B was acetonitrile.

The chromatograph was programmed as follows: Initial: 80% mobile phase A and 20% mobile phase B; 0-20 minutes: linear gradient to 50% mobile phase A; 20-60 minutes: isocratic 50% mobile phase A; and 65-70 minutes: equilibration with 80% mobile phase A.

The chromatograph was equipped with a 280 nm detector, and the flow rate was 1.0 mL per minute at room temperature. Test samples (10 ηL) were prepared by dissolving the appropriate amount of sample in order to obtain 1.0 mg per mL of aqueous phosphoric acid 0.5% (v/v) from HPLC-grade water.

### Example 1

5,6-Dimethoxyindan-1-one (85.0 g), 1-benzylpiperidine-4-carbaldehyde (99.9 g) and potassium hydroxide (19.3 g) were suspended in a mixture of tetrahydrofuran (1250 mL) and water (1250 mL) at room temperature. The mixture was heated to 60° C. and stirred for 7 hours at this temperature. The tetrahydrofuran was then removed from the reaction mixture by distillation. The resulting crystals were isolated by filtration and dried at 60° C. under vacuum to yield 167 g of Compound 1, Form I. (Yield: Quantitative; X-ray powder diffractogram: See FIG. 1).

### Example 2

5,6-Dimethoxyindan-1-one (190 g), 1-benzylpiperidine-4-carbaldehyde (223 g) and potassium hydroxide (43.1 g) were suspended in a mixture of tetrahydrofuran (1200 mL) and water (130 mL) at room temperature. The mixture was heated to reflux temperature and stirred for 8 hours at this temperature. The tetrahydrofuran was then removed from the reaction mixture by distillation while water (1200 mL) was being added. The resulting crystals were isolated by filtration to yield 544 g of Compound 1. (Yield: Quantitative; Loss on Drying: 23.0%, 419 g).

### Example 3

5,6-Dimethoxyindan-1-one (190 g), 1-benzylpiperidine-4-carbaldehyde (223 g) and potassium hydroxide (43.1 g) were suspended in a mixture of tetrahydrofuran (1200 mL) and water (130 mL) at room temperature. The mixture was heated to reflux temperature and stirred for 8 hours at this temperature. The tetrahydrofuran was then removed from the reaction mixture by distillation, and water (1200 mL) was added. The resulting crystals were isolated by filtration to yield 538 g of Compound 1. (Yield: Quantitative; Loss on Drying: 25.5%, 400 g).

### Example 4

5,6-Dimethoxyindan-1-one (60.0 g), 1-benzylpiperidine-4-carbaldehyde (70.5 g), potassium hydroxide (13.6 g) and benzyltriethylammonium chloride (3.56 g) were suspended in a mixture of toluene (380 mL) and water (42 mL) at room temperature. Nitrogen was bubbled through the suspension for 30 minutes, and the mixture was then heated to reflux temperature. After stirring at reflux temperature for 8 hours, water was added (380 mL), and the toluene was removed from the reaction mixture by distillation. The resulting crystals were isolated by filtration to yield 140 g of Compound 1. (Yield: Quantitative; Loss on Drying: 13.3%, 121 g).

### Example 5

5,6-Dimethoxyindan-1-one (30.0 g), 1-benzylpiperidine-4-carbaldehyde (35.2 g), potassium hydroxide (6.81 g) and benzyltriethylammonium chloride (1.78 g) were suspended in a mixture of toluene (190 mL) and water (21 mL) at room temperature. Nitrogen was bubbled through the suspension for 30 minutes, and the mixture was then heated to reflux temperature. After stirring at reflux temperature for 8 hours, water was added (200 mL), and the toluene was removed from the reaction mixture by distillation. The mixture was then cooled to 20-25° C. and the resulting crystals were isolated by filtration to yield 80.4 g of Compound 1. (Yield: 97.8%; Loss on Drying: 28.3%, 57.6 g).

### Example 6

5,6-Dimethoxyindan-1-one (30.0 g), 1-benzylpiperidine-4-carbaldehyde (35.2 g), potassium hydroxide (6.81 g) and benzyltriethylammonium chloride (1.78 g) were suspended in a mixture of toluene (190 mL) and water (20 mL) at room temperature. Nitrogen was bubbled through the suspension for 30 minutes, and the mixture was then heated to reflux temperature. After stirring at reflux temperature for 8 hours, water was added (190 mL), and the toluene was removed from the reaction mixture by distillation. The mixture was then cooled to 20-25° C., and the resulting crystals were isolated by filtration to yield 72.2 g of Compound 1. (Yield: Quantitative; Loss on Drying: 19.0%, 58.5 g).

### Example 7

5,6-Dimethoxyindan-1-one (130 g), 1-benzylpiperidine-4-carbaldehyde (153 g), potassium hydroxide (29.5 g) and benzyltriethylammonium chloride (7.70 g) were suspended in a mixture of toluene (825 mL) and water (92 mL) at room temperature. Nitrogen was bubbled through the suspension for 30 minutes, and the mixture was then heated to reflux temperature. After stirring at reflux temperature for 8 hours, water was added (825 mL), and the toluene was removed from the reaction mixture by distillation. The mixture was then cooled to 20-25° C., and the resulting crystals were isolated by filtration to yield 327 g of Compound 1. (Yield: Quantitative; Loss on Drying: 17.3%, 270 g).

### Example 8

5,6-Dimethoxyindan-1-one (10.0 g), 1-benzylpiperidine-4-carbaldehyde (11.8 g), potassium hydroxide (2.27 g) and benzyltriethylammonium chloride (0.59 g) were suspended in a mixture of toluene (64 mL) and water (3.6 mL) at room temperature. Nitrogen was bubbled through the suspension for 30 minutes, and the mixture was then heated to reflux temperature. After stirring at reflux temperature for 5.5 hours, the mixture was cooled to 20-25° C. The resulting crystals were then isolated by filtration to yield 22.2 g of Compound 1. (Yield: Quantitative; Loss on Drying: 8.42%, 20.3 g).

### Example 9

5,6-Dimethoxyindan-1-one (10.0 g), 1-benzylpiperidine-4-carbaldehyde (11.8 g), potassium hydroxide (2.27 g) and benzyltriethylammonium chloride (0.59 g) were suspended in a mixture of toluene (64 mL) and water (3.6 mL) at room temperature. Nitrogen was bubbled through the suspension for 30 minutes, and the mixture was then heated to reflux temperature. After stirring at reflux temperature for 5.5 hours, the mixture was cooled to 20-25° C. The resulting crystals were then isolated by filtration to yield 25.2 g of Compound 1, Form II. (Yield: Quantitative; Loss on Drying: 9.43%, 22.8 g; X-ray powder diffractogram: See FIG. 2).

### Example 10

5,6-Dimethoxyindan-1-one (20.0 g), 1-benzylpiperidine-4-carbaldehyde (23.5 g), potassium hydroxide (4.54 g) and benzyltriethylammonium chloride (1.19 g) were suspended in a mixture of toluene (128 mL) and water (7.2 mL) at room temperature. Nitrogen was bubbled through the suspension for 30 minutes, and the mixture was then heated to reflux temperature. After stirring at reflux temperature for 5 hours, the mixture was cooled to 20-25° C. The resulting crystals were then isolated by filtration to yield 42.0 g of Compound 1, Form II. (Yield: 93.4%; Loss on Drying: 11.3%, 37.3 g).

### Example 11

5,6-Dimethoxyindan-1-one (13.6 Kg), 1-benzylpiperidine-4-carbaldehyde (16.0 Kg), potassium hydroxide (2.72 Kg) and benzyltriethylammonium chloride (0.82 Kg) were suspended in a mixture of toluene (75 Kg) and water (4.4 Kg) at room temperature. The vessel was inertized with nitrogen/vacuum, and the mixture was then heated to reflux temperature. After stirring at reflux temperature for 5 hours, the mixture was cooled to 20-25° C. The resulting crystals were then isolated by filtration, suspended in water (159 Kg) and stirred at 10° C. for 30 minutes. The suspension was filtered, and the resulting solid was suspended in isopropyl alcohol (128 Kg). The suspension was then heated to reflux temperature for 3 hours and then cooled to 20-25° C. The resulting crystals were then isolated by filtration to yield 27.0 g of Compound 1, Form I. (Yield: 98.09%; Loss on Drying: 3.0%, 26.2 g; X-ray powder diffractogram: See FIG. 1).

### Example 12

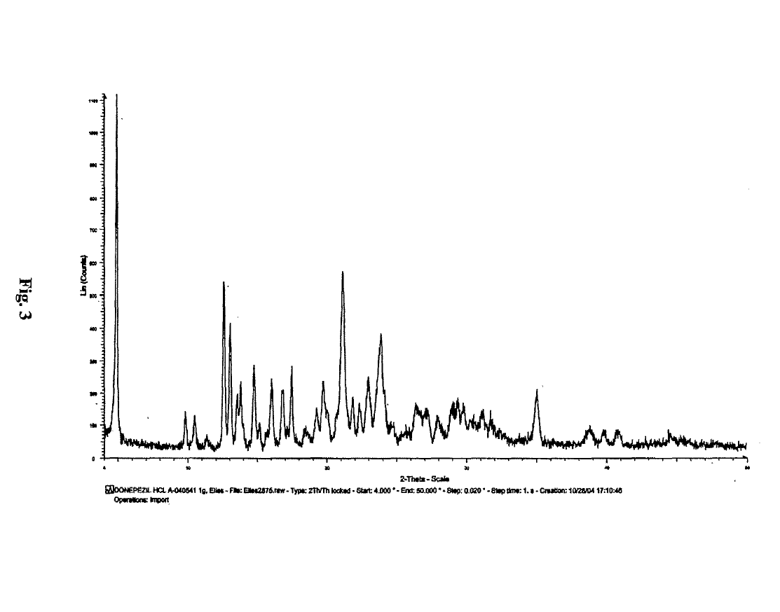

2-(1-benzylpiperidin-4-ylmethyliden)-5,6-dimethoxyindan-1-one (27.0 Kg, obtained in Example 11) and Pt/C catalyst (2.34 Kg, ˜50% H₂O, 5% Pt) were suspended in ethyl acetate (144 Kg). The temperature was set to 20-25° C. The vessel was then inertized with nitrogen and pressurized with hydrogen (1.0 barg). After stirring under these conditions for approximately 7 hours, the vessel was inertized again to remove all hydrogen, the catalyst was removed by filtration, and the vessel was washed with ethyl acetate (54 Kg). The ethyl acetate (about 144 Kg) was then removed by distillation at atmospheric pressure. The remaining solution was then cooled to 45-50° C., and methanol (54 Kg) was added. The solution was then cooled to 20-25° C., and 35% hydrochloric acid (7.2 Kg) and methyl tert-butylether (101 Kg) were added. The mixture was further cooled to 0-5° C. and stirred at this temperature for 1 hour. The resulting solid was isolated by filtration, washed with methyl tert-butylether (10 Kg) and dried at 40° C. for 24 hours to yield 14.00 Kg of donepezil hydrochloride, polymorph I. (Yield: 47.6%; Purity: 99.85%; X-ray powder diffractogram: See FIG. 3).

TABLE 1
Summary of HPLC Purity Data for Examples 1-11*

| Ex- am- ple | 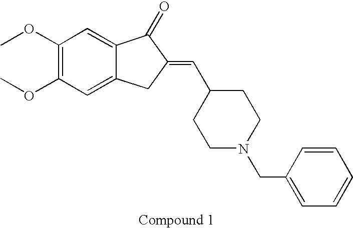 2-(1-Benzylpiperidin-4- ylmethyliden)-5,6-dimethoxyindan-1- one | 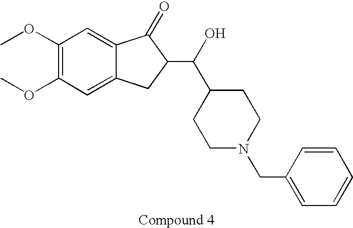 2-[(1-Benzylpiperidin-4- yl)hydroxymethyl]-5,6- dimethoxyindan-1-one | 2-(1-Benzylpiperidin- 4-ylmethyliden)-5,6- dimethoxyindan-1-one isomer |
|---|---|---|---|
| 1 | 95.744 | 0.719 | 1.874 |
| 2 | 95.515 | 0.813 | 2.381 |
| 3 | 95.251 | 1.118 | 2.650 |
| 4 | 92.027 | 0.641 | 3.658 |
| 5 | 94.291 | 2.481 | 2.248 |
| 6 | 94.577 | 1.747 | 2.279 |
| 7 | 93.332 | 0.473 | 2.608 |
| 8 | 98.289 | 0.104 | 1.285 |
| 9 | 97.995 | 0.043 | 1.388 |
| 10 | 98.466 | 0.113 | 1.061 |
| 11 | 98.941 | 0.041 | 0.816 |

*All tabulated data correspond to area % when analyzed by the accompanying HPLC method

Although the invention has been described and illustrated with a certain degree of particularity, it is understood that the disclosure has been made only by way of example, and that numerous changes in the conditions and order of steps can be resorted to by those skilled in the art without departing from the spirit and scope of the invention.

What is claimed is:

1. A process for preparing 2-(1-benzylpiperidin-4-ylmethyliden)-5,6-dimethoxyindan-1-one comprising reacting 5,6-dimethoxyindan-1-one with 1-benzylpiperidine-4-carbaldehyde using an alkali metal hydroxide in a mixture of at least one organic solvent and water at a temperature between room temperature and 120° C.

2. The process of claim 1, further comprising the use of a phase transfer catalyst.

3. The process of claim 1, further comprising the step of isolating said 2-(1-benzylpiperidin-4-ylmethyliden)-5,6-dimethoxyindan-1-one.

4. The process of claim 3, wherein said step of isolating said 2-(1-benzylpiperidin-4-ylmethyliden)-5,6-dimethoxyindan-1-one comprises filtration from water after removing said at least one organic solvent.

5. The process of claim 3, wherein said step of isolating said 2-(1-benzylpiperidin-4-ylmethyliden)-5,6-dimethoxyindan-1-one comprises filtration after cooling said mixture.

6. The process of claim 1, further comprising the step of purifying 2-(1-benzylpiperidin-4-ylmethyliden)-5,6-dimethoxyindan-1-one with at least one purifying solvent.

7. The process of claim 6, wherein said at least one purifying solvent is isopropyl alcohol.

8. The process of claim 6, wherein said at least one purifying solvent is water.

9. The process of claim 1, wherein said alkali metal hydroxide is at least one of potassium hydroxide, sodium hydroxide and combinations thereof.

10. The process of claim 9, wherein said alkali metal hydroxide is potassium hydroxide.

11. The process of claim 1, wherein said organic solvent is at least one of toluene, xylene, tetrahydrofuran, 2-methyltetrahydrofuran and combinations thereof.

12. The process of claim 11, wherein said organic solvent is toluene.

13. The process of claim 11, wherein said organic solvent is tetrahydrofuran.

14. The process of claim 2, wherein said phase transfer catalyst is at least one of benzyltriethyl ammonium chloride, benzyltriethylammonium bromide, tetraethylammonium chloride, tetraethylammonium bromide, tetrabutylammonium chloride, tetrabutylammonium bromide, tributylmethylammonium chloride, tributylmethylammonium bromide, benzyltrimethyl ammonium chloride, benzyltrimethylammonium bromide, tetrahexylammonium chloride, tetrahexylammonium bromide, tetraoctylammonium chloride, tetraoctylammonium bromide, hexadecyltrimethylammonium chlorides, hexadecyltrimethylammonium chloride and combinations thereof.

15. The process of claim 14, wherein said phase transfer catalyst is benzyltriethylammonium chloride.

16. The process of claim 1, wherein said temperature is between approximately 90° C. and approximately 97° C.

17. The process of claim 1, wherein said temperature is at or near reflux.

18. The process of claim 1, further comprising converting said 2-(1-benzylpiperidin-4-ylmethyliden)-5,6-dimethoxyindan-1-one into donepezil hydrochloride.

19. The process of claim 18, wherein said step of converting said 2-(1-benzylpiperidin-4-ylmethyliden)-5,6-dimethoxyindan-1-one into donepezil hydrochloride comprises hydrogenating said 2-(1-benzylpiperidin-4-ylmethyliden)-5,6-dimethoxyindan-1-one and treating the product thereof with hydrochloric acid or an equivalent thereof.

20. A polymorphic form of solid, crystalline 2-(1-benzylpiperidin-4-ylmethyliden)-5,6-dimethoxyindan-1-one, designated Form I, having an X-ray diffraction pattern (2θ) substantially similar to that of FIG. 1.

21. The Form I polymorph of claim 20, wherein said solid, crystalline 2-(1-benzylpiperidin-4-ylmethyliden)-5,6-dimethoxyindan-1-one is prepared according to the process of claim 1.

22. The Form I polymorph of claim 20 having an X-ray diffraction pattern (20) having characteristic peaks at 5.28, 10.52, 11.54, 13.40, 17.51, 18.17, 19.24, 20.24, 20.95, 22.23, 23.15, 24.52, 25.64, 26.16 degrees.

23. The Form I polymorph of claim 20, wherein said solid, crystalline 2-(1-benzylpiperidin-4-ylmethyliden)-5,6-dimethoxyindan-1-one is prepared by (i) reacting 5,6-dimethoxyindan-1-one with 1-benzylpiperidine-4-carbaldehyde in a mixture of toluene and water and (ii) isolating said solid, crystalline 2-(1-benzylpiperidin-4-ylmethyliden)-5,6-dimethoxyindan-1-one by filtration after cooling said mixture of toluene and water.

24. A polymorphic form of solid, crystalline 2-(1-benzylpiperidin-4-ylmethyliden)-5,6-dimethoxyindan-1-one, designated Form II, having an X-ray diffraction pattern (2θ) substantially similar to that of FIG. 2.

25. The Form II polymorph of claim 24, wherein said solid, crystalline 2-(1-benzylpiperidin-4-ylmethyliden)-5,6-dimethoxyindan-1-one is prepared according to the process of claim 1.

26. The Form II polymorph of claim 24 having an X-ray diffraction pattern (2θ) having characteristic peaks at 8.17, 11.51, 14.87, 17.68, 19.29, 19.91,21.09, 21.74, 24.75, 27.62 degrees.

27. The Form I polymorph of claim 20 having a purity higher than 95% as measured by high performance liquid chromatography.

28. The Form I polymorph of claim 20 having a purity higher than 98% as measured by high performance liquid chromatography.

29. The Form I polymorph of claim 20 having a purity higher than 98.9% as measured by high performance liquid chromatography.

30. The Form I polymorph of claim 20 having a content of 2-[(1-benzylpiperidin-4-yl)hydroxymethyl]-5,6-dimethoxyindan-1-one of less than 2.5% as measured by high performance liquid chromatography.

31. The Form I polymorph of claim 20 having a content of 2-[(1-benzylpiperidin-4-yl)hydroxymethyl]-5,6-dimethoxyindan-1-one of less than 1.0% as measured by high performance liquid chromatography.

32. The Form I polymorph of claim 20 having a content of 2-[(1-benzylpiperidin-4-yl)hydroxymethyl]-5,6-dimethoxyindan-1-one of less than 0.5% as measured by high performance liquid chromatography.

33. The Form II polymorph of claim 24 having a purity higher than 95% as measured by high performance liquid chromatography.

34. The Form II polymorph of claim 24 having a purity higher than 98% as measured by high performance liquid chromatography.

35. The Form II polymorph of claim 24 having a purity higher than 98.9% as measured by high performance liquid chromatography.

36. The Form II polymorph of claim 24 having a content of 2-[(1-benzylpiperidin-4-yl)hydroxymethyl]-5,6-dimethoxyindan-1-one of less than 2.5% as measured by high performance liquid chromatography.

37. The Form II polymorph of claim 24 having a content of 2-[(1-benzylpiperidin-4-yl)hydroxymethyl]-5,6-dimethoxyindan-1-one of less than 1.0% as measured by high performance liquid chromatography.

38. The Form II polymorph of claim 24 having a content of 2-[(1-benzylpiperidin-4-yl)hydroxymethyl]-5,6-dimethoxyindan-1-one of less than 0.5% as measured by high performance liquid chromatography.

39. A mixture comprising 2-(1-benzylpiperidin-4-ylmethyliden)-5,6-dimethoxyindan-1-one polymorph Form I and 2-(1-benzylpiperidin-4-ylmethyliden)-5,6-dimethoxyindan-1-one polymorph Form II.

40. The mixture of claim 39, wherein each of 2-(1-benzylpiperidin-4-ylmethyliden)-5,6-dimethoxyindan-1-one polymorph Form I and 2-(1-benzylpiperidin-4-ylmethyliden)-5,6-dimethoxyindan-1-one polymorph Form II has a purity higher than 95% as measured by high performance liquid chromatography.

41. The mixture of claim 39, wherein each of 2-(1-benzylpiperidin-4-ylmethyliden)-5,6-dimethoxyindan-1-one polymorph Form I and 2-(1-benzylpiperidin-4-ylmethyliden)-5,6-dimethoxyindan-1-one polymorph Form II has a purity higher than 98% as measured by high performance liquid chromatography.

42. The mixture of claim 39, wherein each of 2-(1-benzylpiperidin-4-ylmethyliden)-5,6-dimethoxyindan-1-one polymorph Form I and 2-(1-benzylpiperidin-4-ylmethyliden)-5,6-dimethoxyindan-1-one polymorph Form II has a purity higher than 98.9% as measured by high performance liquid chromatography.

43. The mixture of claim 39 having a content of 2-[(1-benzylpiperidin-4-yl)hydroxymethyl]-5,6-dimethoxyindan-1-one of less than 2.5% as measured by high performance liquid chromatography.

44. The mixture of claim 39 having a content of 2-[(1-benzylpiperidin-4-yl)hydroxymethyl]-5,6-dimethoxyindan-1-one of less than 1.0% as measured by high performance liquid chromatography.

45. The mixture of claim 39 having a content of 2-[(1-benzylpiperidin-4-yl)hydroxymethyl]-5,6-dimethoxyindan-1-one of less than 0.5% as measured by high performance liquid chromatography.

46. A process for preparing donepezil hydrochloride comprising converting at least one compound of 2-(1-benzylpiperidin-4-ylmethyliden)-5,6-dimethoxyindan-1-one polymorph Form I, 2-(1-benzylpiperidin-4-ylmethyliden)-5,6-dimethoxyindan-1-one polymorph Form II and mixtures thereof into donepezil hydrochloride.

47. The process of claim 46, wherein said at least one compound is 2-(1-benzylpiperidin-4-ylmethyliden)-5,6-dimethoxyindan-1-one polymorph Form I.

48. The process of claim 46, wherein said at least one compound is 2-(1-benzylpiperidin-4-ylmethyliden)-5,6-dimethoxyindan-1-one polymorph Form II.

49. The process of claim 46, wherein said at least one compound is a mixture of 2-(1-benzylpiperidin-4-ylmethyliden)-5,6-dimethoxyindan-1-one polymorph Form I and 2-(1-benzylpiperidin-4-ylmethyliden)-5,6-dimethoxyindan-1-one polymorph Form II.

50. Donepezil hydrochloride prepared according to the process of claim 46.

51. Donepezil hydrochloride prepared according to the process of claim 47.

52. Donepezil hydrochloride prepared according to the process of claim 48.

53. Donepezil hydrochloride prepared according to the process of claim 49.

54. A process for preparing 2-(1-benzylpiperidin-4-ylmethyliden)-5,6-dimethoxyindan-1-one comprising reacting 5,6-dimethoxyindan-1-one with 1-benzylpiperidine-4-carbaldehyde using potassium hydroxide in a mixture of toluene and water at reflux temperature.

55. The process of claim 54, further comprising the use of a phase transfer catalyst.

56. The process of claim 54, further comprising the step of isolating said 2-(1-benzylpiperidin-4-ylmethyliden)-5,6-dimethoxyindan-1-one.

57. The process of claim 56, wherein said step of isolating said 2-(1-benzylpiperidin-4-ylmethyliden)-5,6-dimethoxyindan-1-one comprises filtration from water after removing said toluene.

58. The process of claim 56, wherein said step of isolating said 2-(1-benzylpiperidin-4-ylmethyliden)-5,6-dimethoxyindan-1-one comprises filtration after cooling said mixture.

59. The process of claim 54, further comprising the step of purifying 2-(1-benzylpiperidin-4-ylmethyliden)-5,6-dimethoxyindan-1-one with at least one purifying solvent.

60. The process of claim 59, wherein said at least one purifying solvent is isopropyl alcohol.

61. The process of claim 59, wherein said at least one purifying solvent is water.
